(19) United States
(12) Patent Application Publication
(10) Pub. No.: US 2021/0125152 A1
(43) Pub. Date: Apr. 29, 2021

(54) PET CARE MANAGEMENT SYSTEMS AND METHODS

(71) Applicant: Figo Pet Insurance, LLC, Chicago, IL (US)

(72) Inventors: Howard E. Sproat, Hefei (CN); James Aubry, Chicago, IL (US); Javier Solorio, Palatine, IL (US); Manish Kapoor, Uttar Pradesh (IN)

(21) Appl. No.: 17/083,018

(22) Filed: Oct. 28, 2020

Related U.S. Application Data
(60) Provisional application No. 63/035,195, filed on Jun. 5, 2020.
(60) Provisional application No. 62/926,774, filed on Oct. 28, 2019.

(51) Int. Cl. G06Q 10/10; G16H 40/20; G16H 10/60

(52) U.S. Cl. CPC G06Q 10/1093; G16H 10/60; G16H 40/20

(57) ABSTRACT

A pet care management system for scheduling a pet care appointment based on a medical record for a pet includes a user device including a camera subsystem, a database storing a calendar associated with the pet, a processor in communication with the user device and the database, and memory. The processor is configured to capture, via the camera subsystem on the user device, image data representing the medical record related to a pet health care provider appointment, wherein the image data includes a date associated with veterinary services provided to the pet; determine an appointment date for a subsequent appointment based on the date associated with veterinary services; and automatically populate the calendar with the subsequent appointment on the appointment date.

### CROSS-REFERENCE TO RELATED APPLICATIONS

This application incorporates by reference and claims the benefit of priority to U.S. Provisional Application No. 63/035,195 filed Jun. 5, 2020 and U.S. Provisional Application No. 62/926,774 filed Oct. 28, 2019.

### BACKGROUND OF THE INVENTION

The present subject matter relates generally to a multifunctional platform for pet owners.

Conventionally, pet owners looking for other pet owners typically have to meet them by chance, perhaps at a dog park or from seeing a photo on a coworker's desk. To find pet friendly establishments, a pet owner would have to search on the internet and find individual establishments that allow pets, write them down, find the hours of operation, find the address, map out directions, etc. Further, pet owners also must rely on individual internet searches to find pet events. As a result, there is a need for a service that provides a single platform for connecting pet owners to each other, pet businesses, pet friendly establishments, and pet events.

Veterinarian appointments are expensive and time consuming. As a result, many users avoid an official appointment and seek unprofessional advice from the internet. The user's veterinarian office may be booked for months at a time, making obtaining an appointment in the near future difficult. The user cannot seek a sooner appointment from a different veterinarian in the user's location because all of the pet's medical records are held by the original veterinarian office of the pet that cannot see the pet for weeks. Thus, there is a need for veterinarian access for a user that is immediate and provides the veterinarian access to the pet's medical history.

Health records for animals are typically maintained by a veterinarian office describing the medical history of the animal including the immunizations, past infections, past test results, etc. Alternatively, when a user has to move location, the user has to print out all the medical records and deliver them to a new veterinarian office in the user's new location. Thus, there is a need for a single platform to maintain a user's pet's medical documents that can be accessed by the user and any veterinarian office or other pet business.

Many pet owners implant microchips on their pets in order to track pets in the event that they are lost. However, many pet owners often lose track of the microchip information, including the microchip number and the login information. After many years, owners tend to forget the company that supplied the microchip, leaving the owner with no way to update the information.

### BRIEF SUMMARY OF THE TECHNOLOGY

The present disclosure provides a multifunctional platform for pet owners. The pet care management system may include a centralized server, one or more database(s), and an end user device. The centralized server may include a processor and memory, with the processor executing computer code stored in the memory. The end user device may run a stand-alone end user device application.

The pet care management system displays a series of graphical user interfaces on the user device that enables interaction between a plurality of modules including at least one of a communications module, a calendar module, a document module, a pet insurance module, a veterinarian module, a payment module, a recommendations module, and a social network module. The user device also includes a camera subsystem that captures images that can be stored on the device and/or on the database. Various examples of the systems and methods are provided herein.

The system is a dynamic platform that continually analyzes user and pet data (e.g., profile information, medical records, prescriptions, live movement data, user movement data, historical data, etc.) to proactively generate notifications, appointments, insurance claims, and coordination of the pet life.

In one embodiment, the pet care management system for scheduling a pet care appointment based on a medical record for a pet is provided. The pet management system includes a user device including a camera subsystem, a database storing a calendar associated with the pet, a processor in communication with the user device and the database, and memory in communication with the processor. The memory stores computer executable instructions such that, when the instructions are executed by the processor, the processor is configured to: capture, via the camera subsystem on the user device, image data representing the medical record related to a pet health care provider appointment, wherein the image data includes a date associated with veterinary services provided to the pet; determine an appointment date for a subsequent appointment based on the date associated with veterinary services; and automatically populate the calendar with the subsequent appointment on the appointment date.

In another embodiment, a method for scheduling a pet care appointment based on a medical record for a pet through a user device is provided. The method includes the steps of: providing camera functionality through a graphical user interface to the user device; providing a database storing a calendar associated with the pet; capturing, via a camera subsystem on the user device, image data representing the medical record related to a pet health care provider appointment, wherein the image data includes a date associated with veterinary services provided to the pet; determining an appointment date for a subsequent appointment based on the date associated with veterinary services; and automatically populating the calendar with the subsequent appointment on the appointment date.

An advantage of the system is providing a document management subsystem that can receive, store, and create documents related to a user's pet. The documents can be any form including medical records, images, among others. The system can use text and image recognition methods to automatically extract relevant data and store and use in appropriate modules within the system.

The system can also include a communication platform (e.g., email server, video imaging, audio, among others) to provide communications directly to other pet owners in a user's social network, veterinarians, businesses, among others. The communication system can allow a user or the system to deliver medical records to a veterinarian, a pet day care, dog walker, etc.

An advantage of the present system is providing a platform to connect pet owners, share photos, plan play dates, and schedule events.

A further advantage of the present system is providing a platform that allows users to explore pet friendly places and services such as pet parks, hiking trails, day care, restaurants, pet walkers, pet groomers, among others.

Another advantage of the present system is communicating notifications to pet owners for pet's birthdays, vaccine due dates, scheduled appointments, scheduled play dates, new pet profiles in the area, predicted weather for usual walk times, among others.

Another advantage of the present system is providing live access to a licensed veterinarian anytime and anywhere. For example, the system can provide a live text, audio, and/or video chat with a veterinarian. As a result, the present system can avoid excessive trips to the veterinarian, thereby reducing costs and time.

Another advantage of the present system is providing a pet insurance platform in communication with the veterinarian business, pet documents, and the user.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a schematic diagram of the components of the business software system of the pet care management system of the present application.

FIG. 2 is a block diagram of a mobile device that may embody the systems and methods described herein.

FIGS. 3-5 are portions of an exemplary home user interface of the pet care management system of FIG. 1.

FIG. 6 is an exemplary reminders user interface of the pet care management system of FIG. 1.

FIG. 7 is a flowchart illustrating an exemplary calendaring method implemented by the pet care management system of FIG. 1.

FIG. 8 is an exemplary documents user interface of the pet care management system of FIG. 1.

FIG. 9 is an exemplary claims user interface of the pet care management system of FIG. 1.

FIGS. 10A-10E illustrate the chat feature of the pet insurance module of the pet care management system of FIG. 1.

FIG. 11 is an exemplary vet user interface of the pet care management system of FIG. 1.

### DETAILED DESCRIPTION

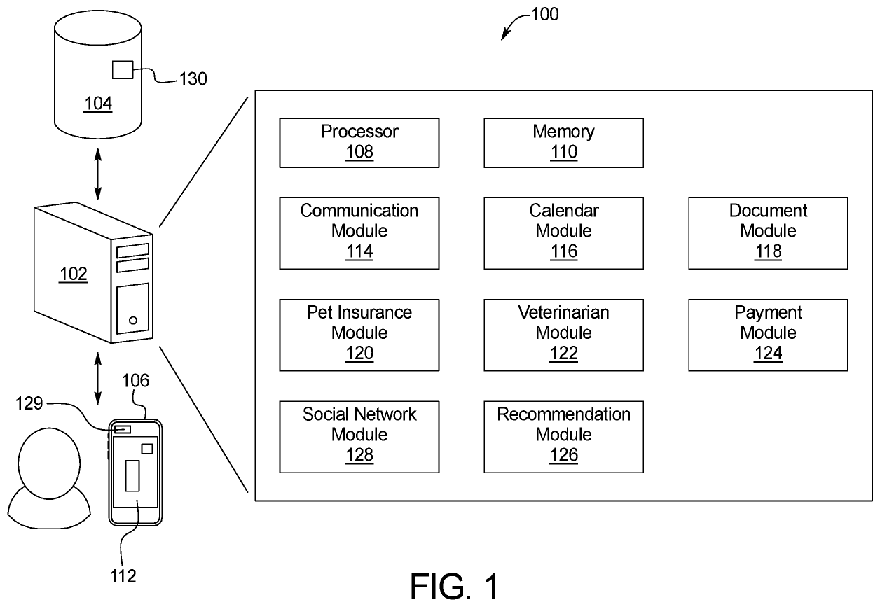

Referring to FIG. 1, the present disclosure provides a pet care management system 100 for managing pet care of a pet. The system provides a single platform for pet owners, veterinarians, and social network connections to access to information related to the user's pet including, for example, the pet's medical records, businesses, location, and events, among others.

The pet care management system 100 may include a centralized server 102, one or more database(s) 104, and an end user device 106. The centralized server 102 may include a processor 108 and memory 110, and the processor 108 may act as a centralized controller that functions to execute computer code stored in the memory 110 of the server 102. The databases 104 may be external or internal to the system 100 and communicate with the centralized server 102 to store and provides data to and from the server 102.

The end user device 106 may run a stand-alone end user device application 112 or access a hosted, internet/intranet accessible website, each of which enable control over the system. In the embodiments illustrated herein, the pet care management system 100 displays a series of graphical user interfaces on the user device 106 that enables interaction between a plurality of modules including at least one of a communications module 114, a calendar module 116, a document module 118, a pet insurance module 120, a veterinarian module 122, a payment module 124, a recommendations module 126, and a social network module 128. Data 130 is stored on and accessed from the database 104 and/or the user device 106. The user device 106 also includes a camera subsystem 129 that captures images that can be stored on the device 106 and/or on the database 104.

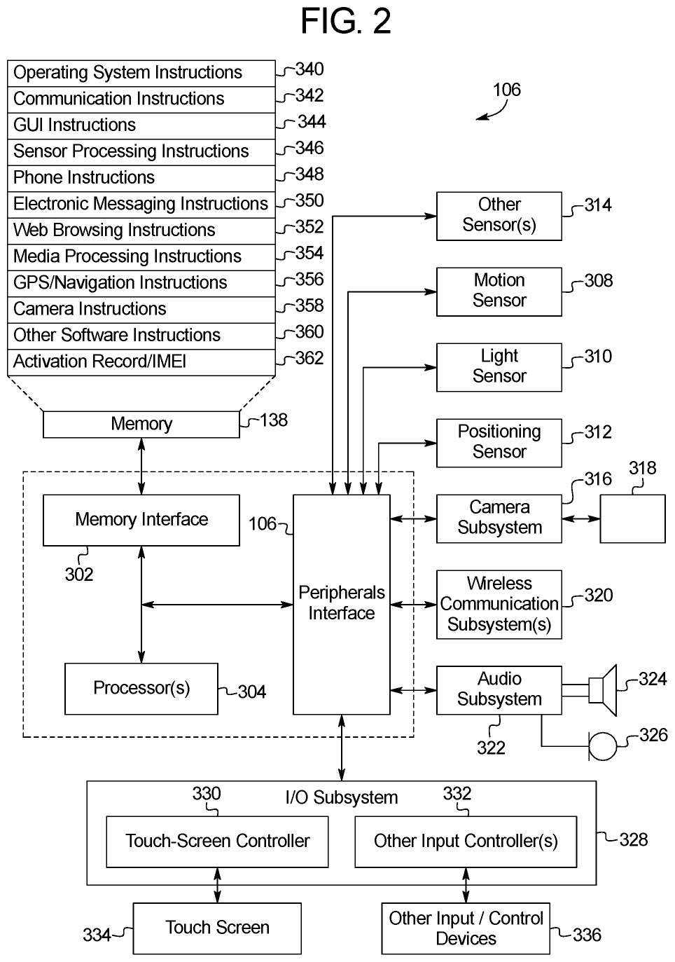

FIG. 2 is a block diagram of an example implementation of a mobile device 106. Referring to FIG. 1, the mobile device 106 includes a memory interface 302, one or more data processors, image processors and/or central processors 304, and a peripherals interface 306. The memory interface 302, the one or more processors 304 and/or the peripherals interface 306 can be separate components or can be integrated in one or more integrated circuits. The various components in the mobile device 106 can be coupled by one or more communication buses or signal lines, as will be recognized by those skilled in the art.

Sensors, devices, and additional subsystems can be coupled to the peripherals interface 306 to facilitate various functionalities. For example, a motion sensor 308 (e.g., a gyroscope), a light sensor 310, and a positioning sensor 312 (e.g., GPS receiver) can be coupled to the peripherals interface 106 to facilitate the orientation, lighting, and positioning functions described further herein. Other sensors 314 can also be connected to the peripherals interface 306, such as a proximity sensor, a temperature sensor, a biometric sensor, or other sensing device, to facilitate related functionalities.

A camera subsystem 316 and an optical sensor 318 (e.g., a charged coupled device (CCD) or a complementary metal-oxide semiconductor (CMOS) optical sensor) can be utilized to facilitate camera functions, such as recording photographs and video clips.

Communication functions can be facilitated through one or more wireless communication subsystems 320, which can include radio frequency receivers and transmitters and/or optical (e.g., infrared) receivers and transmitters. The specific design and implementation of the communication subsystem 320 can depend on the communication network(s) over which the mobile device 106 is intended to operate. For example, the mobile device 106 can include communication subsystems 320 designed to operate over a GSM network, a GPRS network, an EDGE network, a Wi-Fi or WiMax network, and a Bluetooth™ network. In particular, the wireless communication subsystems 320 may include hosting protocols such that the mobile device 106 may be configured as a base station for other wireless devices.

An audio subsystem 322 can be coupled to a speaker 324 and a microphone 326 to facilitate voice-enabled functions, such as voice recognition, voice replication, digital recording, and telephony functions.

The I/O subsystem 328 can include a touch screen controller 330 and/or other input controller(s) 332. The touch-screen controller 330 can be coupled to a touch screen 334. The touch screen 334 and touch screen controller 330 can, for example, detect contact and movement, or break thereof, using any of a plurality of touch sensitivity technologies, including but not limited to capacitive, resistive, infrared, and surface acoustic wave technologies, as well as other proximity sensor arrays or other elements for determining one or more points of contact with the touch screen 334. The other input controller(s) 332 can be coupled to other input/control devices 336, such as one or more buttons, rocker switches, thumb-wheel, infrared port, USB port, and/or a pointer device such as a stylus. The one or more buttons (not shown) can include an up/down button for volume control of the speaker 324 and/or the microphone 326.

The memory interface 302 can be coupled to memory 338. The memory 338 can include high-speed random access memory and/or non-volatile memory, such as one or more magnetic disk storage devices, one or more optical storage devices, and/or flash memory (e.g., NAND, NOR). The memory 338 can store operating system instructions 340, such as Darwin, RTXC, LINUX, UNIX, OS X, iOS, WINDOWS, or an embedded operating system such as VxWorks. The operating system instructions 340 may include instructions for handling basic system services and for performing hardware dependent tasks. In some implementations, the operating system instructions 340 can be a kernel (e.g., UNIX kernel).

The memory 338 may also store communication instructions 342 to facilitate communicating with one or more additional devices, one or more computers and/or one or more servers. The memory 338 may include graphical user interface instructions 344 to facilitate graphic user interface processing; sensor processing instructions 346 to facilitate sensor-related processing and functions; phone instructions 348 to facilitate phone-related processes and functions; electronic messaging instructions 350 to facilitate electronic-messaging related processes and functions; web browsing instructions 352 to facilitate web browsing-related processes and functions; media processing instructions 354 to facilitate media processing-related processes and functions; GPS/Navigation instructions 356 to facilitate GPS and navigation-related processes and instructions; camera instructions 358 to facilitate camera-related processes and functions; and/or other software instructions 360 to facilitate other processes and functions (e.g., access control management functions, etc.). The memory 338 may also store other software instructions (not shown) controlling other processes and functions of the mobile device 106 as will be recognized by those skilled in the art In some implementations, the media processing instructions 354 are divided into audio processing instructions and video processing instructions to facilitate audio processing-related processes and functions and video processing-related processes and functions, respectively. An activation record and International Mobile Equipment Identity (IMEI) 362 or similar hardware identifier can also be stored in memory 238.

Each of the above identified instructions and applications can correspond to a set of instructions for performing one or more functions described herein. These instructions need not be implemented as separate software programs, procedures, or modules. The memory 338 can include additional instructions or fewer instructions. Furthermore, various functions of the mobile device 106 may be implemented in hardware and/or in software, including in one or more signal processing and/or application specific integrated circuits.

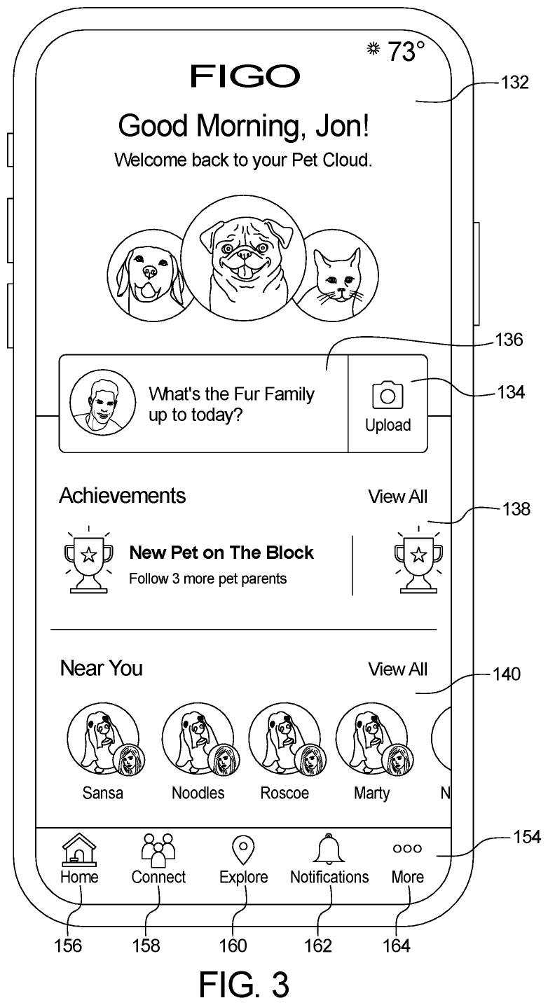

FIGS. 3-10 illustrate example embodiments of graphical user interfaces (GUIs) displayed through the mobile application 112 of the pet care management system 100. Referring to FIGS. 3-5, the illustrated home user interface 132 includes profile information as well as buttons that enable access to each module. The home user interface 132 allows the user to update profile information, such as a button 134 to add photos and a status update input field 136 for updating the user's status in the social media module 128. The home user interface 132 also provides a listing of achievements 138 and a listing of nearby pets 140 currently located near the user, each listing 138, 140 including buttons that lead the user to additional information in additional user interfaces. In some embodiments, the user can direct message or post text messages and images to the friend's feed by selecting one of the users listed. In other embodiments, the home GUI 132 may include a feed of the most recent posts of the user's contacts and/or friends via the social network module 128.

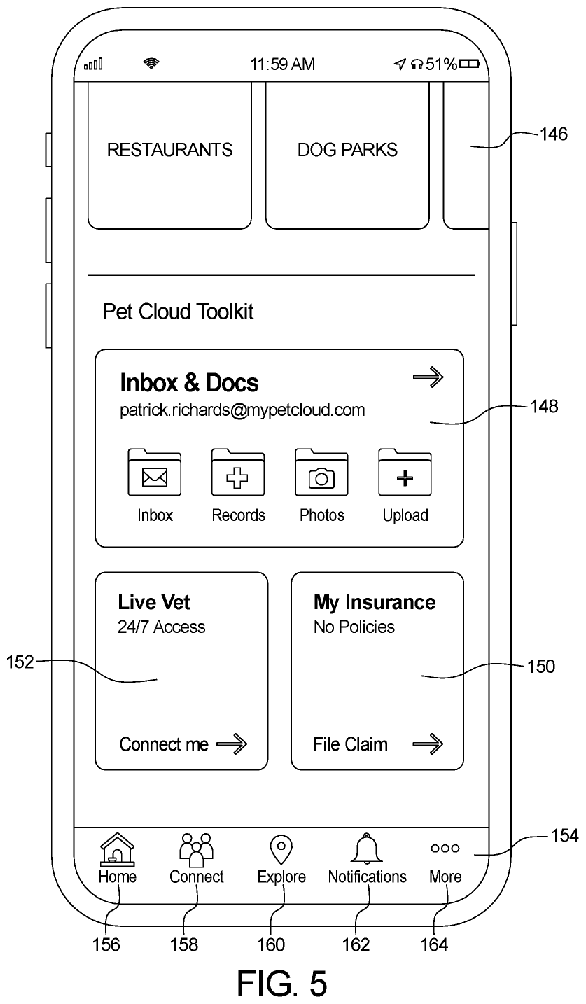

The home GUI 132 also provides a listing of reminders 142 for upcoming events along with a button 144 that leads users to a GUI for creating a new event and a button to view all reminders in the calendar module 116. The home user interface 132 also provides links 146 to popular locations, such as restaurants, dog parks, pet day cares, boarders, pet stores, and groomers. Referring to FIG. 5, the home user interface also includes buttons 148, 150, 152 that, when selected, lead the user to the documents user interface 188, the live vet user interface, and the insurance user interface.

The profile information within the pet care management system 100 enables the user to organize and maintain information for each pet. Each user maintains a user profile with as many pet profiles as needed. The user completes a pet profile for each pet, listing one or more vets, the insurance policy, and the microchip information, among other details.

In one embodiment, the system 100 receives a photo of the user's pet and automatically prepopulates a profile for the user pet based on image analysis. For example, the user can take a photo of the pet through the camera system 129 of the system 100 and/or upload a photo from the user device 106. The system 100 can prompt the user to correct any prepopulated data entered into the pet profile. In an example, if the user does not know the breed or background of the pet, the system 100 can use the image analysis to determine the breed. Alternatively, or in addition to, the system 100 can use a DNA analysis company to determine the pet breed and background.

In some embodiments, the system 100 assists the user in managing information related to pet microchips. It is common for a pet owner to have a microchip implanted into the pet for purposes of identifying lost pets and looking up the contact information to reunite the lost pet with the pet owner. For these systems to run effectively, the information related to the microchip must be up to date, typically through a web portal provided by the company responsible for the microchip. However, pet owners often forget to update this information and log in to the web portal so infrequently, they may lose their log in information or even forget which company they used for the microchip. Accordingly, the system 100 may provide the user with scheduled reminders to update the information associated with the microchip. In addition, the system 100 may provide the user with reminders to update the information associated with the microchip in response to specific events, such as the input of new contact information by the user in the system 100. For example, if the user changes the address information associated with the user account in the system 100, the system 100 may prompt the user to also update the address information with the microchip provider.

Further complicating the process for updating information related to microchips, the web portals through which users log in and update their information often change over the years in which the information should be kept up to date. In some embodiments, the system 100 keeps a record for the user of the login information used to update the microchip provider system. The record may include user login details (login name and password) as well as the location for the login (the specific URL for logging in to the microchip provider system). The system 100 can periodically review the login procedure for each identified microchip provider and update the location for the login in each associated user account in the system 100 so that the system 100 provides all of the details necessary for a user to login and update the information associated with the microchip, even if the web portal or the login procedures change.

By tracking and updating the details related to the login procedure with each third-party microchip provider, in some embodiments of the system 100, the system 100 can automatically update the user contact information with the third-party microchip provider in response to an update of user contact information in the system 100. For example, in response to a user updating address information in the system 100, the system 100 may provide an auto-login bot to automatically use the information stored in the system 100 to log in to the third-party provider website and automatically update the information in the third-party site using the information in the system 100. Accordingly, a user of the system 100 may automatically keep the microchip provider system up to date simply by keeping the system 100 up to date.

Referring back to FIGS. 3-5, a menu bar 154 is provided at the bottom of the GUIs throughout the mobile application 112. The menu bar 154 features buttons 156-164 that, when selected, lead the user to the home user interface 132, social network user interface, the recommendations user interface, the notifications user interface, and a menu user interface. In one example, the menu user interface includes buttons that lead the user to GUIs related to the calendar module 116, the documents module 118, the pet insurance module 120, the veterinarian module 122, and the payment module 124. In some embodiments, the buttons located in the menu bar 154 and the menu user interface are customizable and selected by the user. In other embodiments additional or fewer buttons may be shown.

Within the calendar module 116, the pet care management system 100 maintains a calendar for each user that enables the user to track vet appointments, vaccinations scheduling, and other milestones. The calendar module 116 can receive and store a user's appointments such as veterinarian visits, pet day care, pet walking, pet boarding businesses, and pet's profile information (e.g., pet birthdays). The calendar module 116 also provides notifications to the user related to pet's birthdays, vaccination deadlines dates, invoice due dates, scheduled appointments, and scheduled play dates, among other modules.

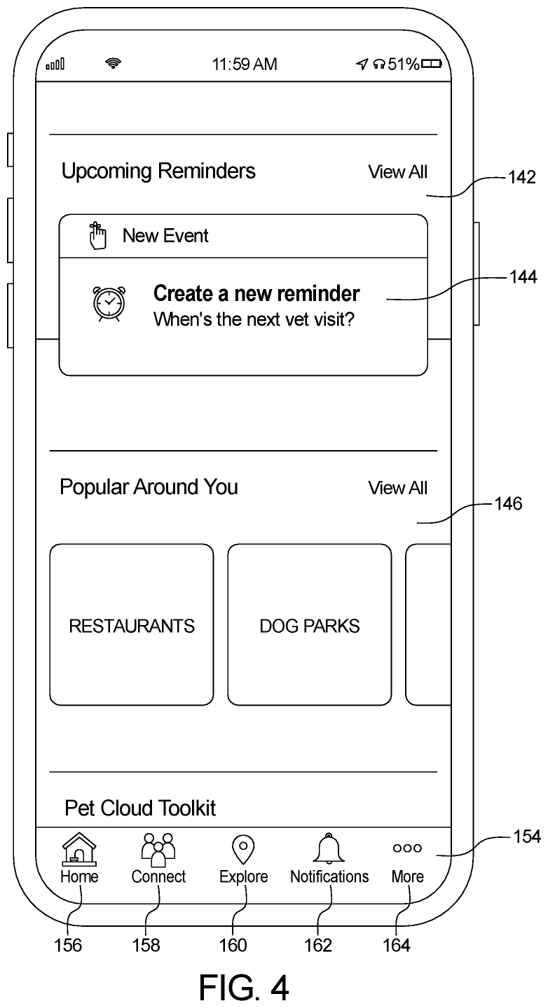

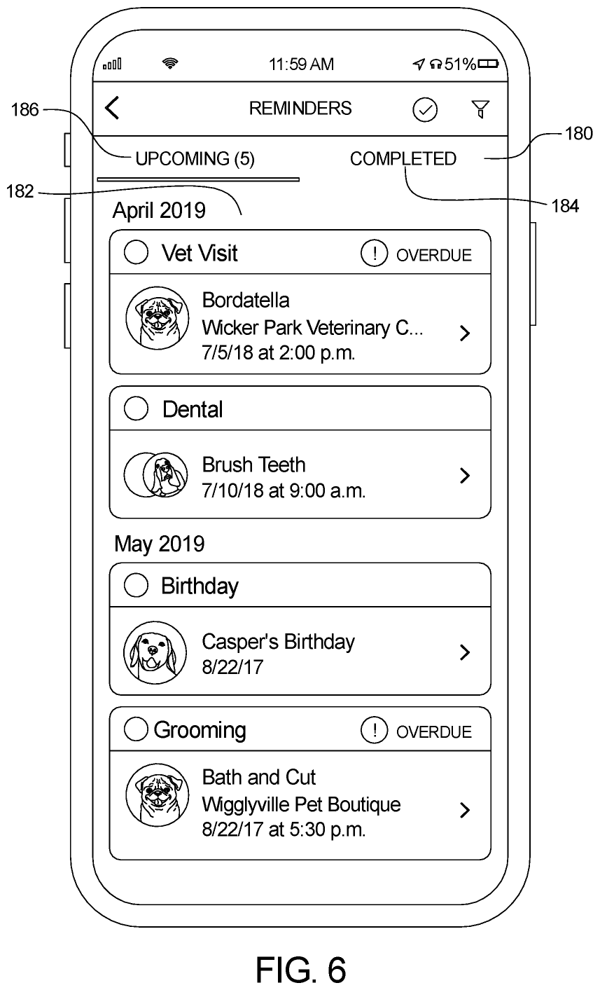

FIG. 6 illustrates a reminder user interface 180 that lists upcoming events 182 or past events, providing buttons 184, 186 that enable the user to toggle between the upcoming events list 182 or the past events list. The calendar module also communicates reminders to other modules as well as the home GUI 132, as shown in FIG. 4.

The calendar is automatically populated with appointments scheduled in other modules, such as the social media module or the veterinarian module. In an example, if a user establishes a play date with a friend in the user's social network in the social network module, the system 100 can automatically upload the appointment from the social network module to the calendar module. Similarly if the user confirms the scheduling of a visit with a veterinarian in the veterinarian module, the visit is automatically scheduled in the calendar within the calendar module 116.

The system 100 also may automatically generate calendar appointments based on data detected or generated from medical records submitted to the documents module 118. As described in greater detail below, the system 100 may use optical character recognition (OCR) to extract data from a document, or convert a document to a machine-readable text. The document may be any suitable medical record including veterinary services such as but not limited to a veterinarian visit record, a vaccination certificate, a vaccination history, a medication history, a surgery record, a hospital visit record, an insurance claim, and a veterinarian invoice.

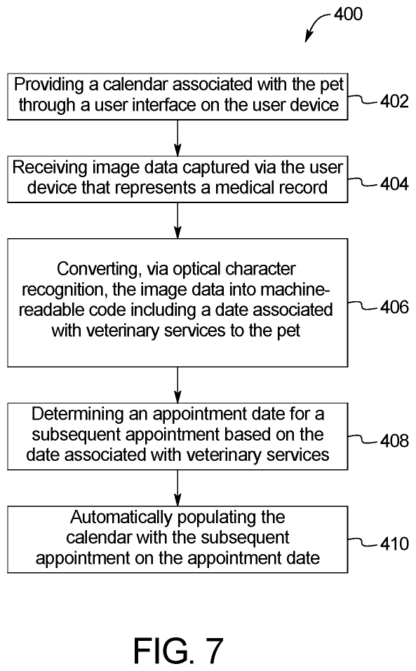

An exemplary method 400 for scheduling a pet care appointment based on a medical record for a pet through a user device is illustrated in FIG. 7. In a first step 402, the system 100 access a calendar associated with the pet that is stored on the database 104. The calendar is maintained in the calendar module 118.

In step 404, the system 100 receives image data from the user device 104 that is captured through the camera functionality or subsystem 129 on his user device 106. The user takes a photo of a medical record related to a pet health care provider appointment, thereby capturing image data representing the medical record, and uploads or enters it into the system 100. The medical record and associated image data includes a date associated with veterinary services provided to the pet.

This image data is then converted to machine-readable code within the system 100 via OCR in step 406. As the image data included a date, the machine-readable code may also include a date on which veterinary services were provided to the pet. For example, the medical record may be a vaccination history showing dates associated with different vaccinations, or the medical record may be a documentation of a veterinary visit to address an ear infection of a cat.

The present system 100 then determines an appointment date for a subsequent appointment based on the date associated with the veterinary services in step 408. The system 100 then automatically populates the calendar with the subsequent appointment on the appointment date in step 410.

In one embodiment, the database 104 includes a plurality of time frames and a plurality of veterinary services types, and one of the plurality of time frames is associated with each one of the plurality of veterinary services types. The system 100 determines the appointment date for the subsequent appointment by identifying a veterinary services type associated with the veterinary services and selecting the appointment date of the subsequent appointment by adding the time frame associated with the veterinary services type of the veterinary services to the date associated with the veterinary services. Each time frame may be a preset number of weeks, months, or years.

Example veterinary services types include a rabies vaccination, a hepatitis vaccination, a canine parvovirus vaccination, a canine distemper vaccination, a Bordetella vaccination, a canine influenza vaccination, a leptospirosis vaccination, and a lyme vaccination. Alternatively, the veterinary services types may include a feline panleukopenia vaccination, feline viral rhinotracheitis vaccination, feline caliciviral disease vaccination, a rabies vaccination, a feline chlamydiosis vaccination, a feline leukemia virus vaccination, a feline infection peritonitis vaccination, a bordetellosis vaccination, a ringworm vaccination, and a giardiasis vaccination. Further, non-limiting examples of veterinary services types include an ear infection, a behavioral consultation, dental care, parasite control, and neutering. Non-limiting examples of veterinary services include a procedure, a date of surgery, a date of treatment, a date of an incident related to the health of the pet, and a date of release from a pet hospital.

In other embodiments, the medical record and associated machine-readable code includes a follow-up appointment date associated with the date associated with veterinary services. The system 100 then simply sets the appointment date of the subsequent appointment based on the follow-up appointment date identified in the medical record and/or machine-readable code. In still further embodiments, the medical record and associated machine-readable code includes a follow-up appointment time frame associated with the date associated with veterinary services. The system 100 then determines the appointment date of the subsequent appointment by adding the follow-up appointment time frame to the date associated with the veterinary services.

For example, a user takes his pet dog to the veterinarian health clinic for a canine influenza vaccination. The veterinarian clinic provides the user with a vaccination certificate as the user leaves the clinic. The user uses the camera feature 129 on the user device 106 to scan the vaccination certificate and stores a copy of the vaccination certificate on the database 104 via the document module. The system 100 converts the vaccination certificate to machine-readable code and identifies a date associated with the canine influenza vaccination. The database 104 includes a plurality of time frames associated with various veterinary services types, and the time frame associated with the canine influenza vaccination is one year. The calendar module then determines an appointment date for a subsequent appointment that is one year from the date of the date in the vaccination certificate that the pet dog received the canine influenza vaccination.

In another example, a user takes his pet cat to the veterinarian health clinic to treat an ear infection. Towards the end of the visit, the veterinarian completes the pet care forms and notes that the user should bring the cat back to the health clinic for a follow-up appointment in two weeks. The clinic provides the user with a veterinarian visit record as he leaves the clinic. The user captures an image of the veterinarian visit record using the camera 129 on his user device 106, and the image data of the veterinarian visit record is stored on the database 104. The system 100 also converts the image data into machine readable code and determines that the machine readable code identifies the date of the visit as the date associated with the ear infection veterinary services, and that the machine readable code includes a follow-up appointment time frame of two weeks. The system 100 determines an appointment date for a subsequent appointment by adding two weeks to the date of the visit, and automatically enters it into the calendar of the user.

The calendar module 116 also generates notifications and reminders related to the appointment date of the subsequent appointment that appear on the home GUI 132 and in the reminder and/or notification user interface. The system 100 may communicate with a scheduling feature of the veterinarian health clinic such that appointment dates of subsequent appointments are automatically scheduled. The system 100 may also provide appointment information to external calendars, such as Google® calendar or iCalendar.

In a still further example, the user may schedule the follow-up appointment as he is leaving the clinic. The veterinarian visit record includes the subsequent appointment, and when the veterinarian visit record is converted to machine-readable code, the system 100 determines that the subsequent appointment has been scheduled. The appointment date for a subsequent appointment is automatically entered into the calendar of the user.

In some embodiments, the medical record and associated machine-readable code may include payment information. The system 100 may identify the payment information and process such information the payment module 124. For example, the user may capture an image of an invoice from a veterinarian clinic, which is then converted to machine-readable code. The payment module 124 may provide notifications to the user via the application 112 that payment is due for the invoice.

The calendar module 116 may include an appointment tool that enables a user to schedule an appointment with a veterinarian, a groomer, etc., without calling or otherwise communicating with the desired business outside of the pet care management system 100. The system 100 can communicate directly with the business for the appointment. In an example, the system 100 may receive reminder communications that a pet is due for a grooming appointment through the communication module or the receipt of a communication in the inbox of the document module 118. The system 100 may automatically schedule an appointment without user input based on the user and pet data. In other examples, the system 100 may automatically schedule an appointment based on the user calendar, a pet medical issue, a pet medical prescription, etc.

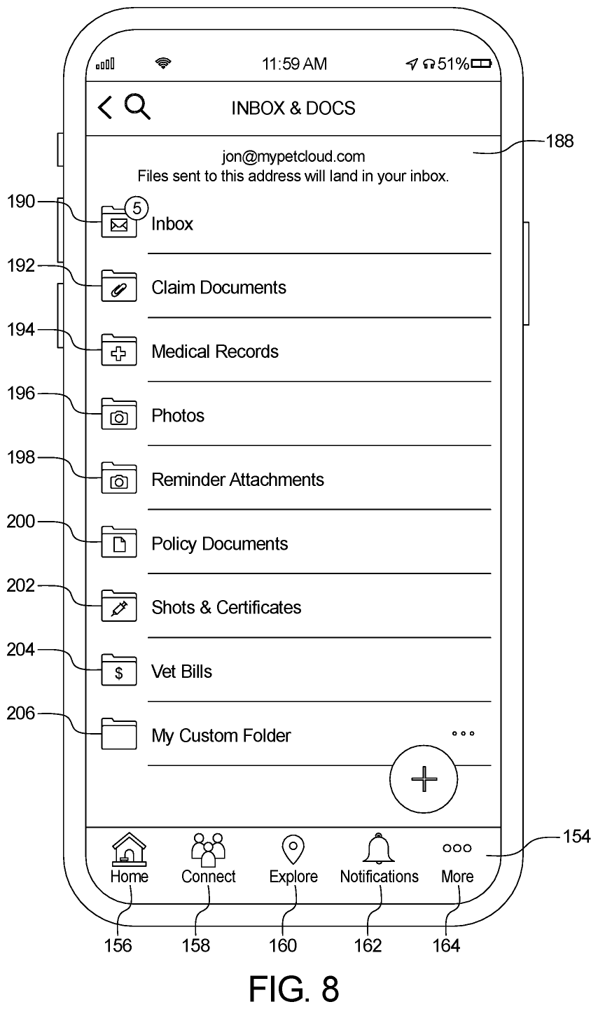

The pet care management system 100 provides a document module 118 that can receive, store, and create documents related to a user's pet. The documents can be any form including medical records, certifications, breeder documents, adoption documents, and images, among others. The documents may be organized into folders as shown in the documents user interface 188 of FIG. 8. Example folders include an inbox folder 190, a claim documents folder 192, a medical records folder 194, a photos folder 196, a reminder attachments folder 198, a policy documents folder 200, a shots and certificates folder 202, a veterinary bills folder 204, and any number of custom folders 206.

The user may capture images of the documents using the camera function 129 of the user device and upload the documents directly to the database 104, or the system can automatically upload documents received in the communication module as documents are processed in other modules such as the pet insurance module, the payment module, or the veterinarian module.

As noted above, the pet care management system 100 can employ optical character recognition (OCR) to scan and extract information from the stored documents and related data or convert the documents into machine-readable code. For example, a user may capture an image of the document, and the documents module may extract data from the document (i.e., convert the document to machine-readable code) via OCR.

The pet care management system 100 may employ OCR to extract the prescription data from the veterinarian bill or medical documents, causing the system 100 to automatically communicate the prescription to a pharmacy. In an example, the system may identify the prescription needed from the medical document and can identify the user's pharmacy from information from the medical document, as previously defined as the selected pharmacy for prescriptions, or from the pet insurance module 120 or other documents within the database 104. The system 100 then communicates the prescription information to the pharmacy associated with the user. Alternatively, the system 100 could determine the location of the user via a GPS tracker within the user device 106 and communicate the prescription information to the nearest pharmacy. The system 100 can send the prescription to either a pharmacy closest to the user's current location and/or a pharmacy closest to the user's previously defined preferred location (e.g., closest to the user's work location, home location, daycare location, etc.).

The system 100 can auto-populate the internal calendar module of the system and/or an external calendar on the user device with the prescription adherence information based on an uploaded or received document, such as image data of a veterinarian bill captured and uploaded by the user or a veterinarian bill emailed to the user. For example, the system can auto-populate reminders and notifications based on the prescription instructions from the veterinarian, the veterinarian invoice, or uploaded from the pharmacy. For example, if the veterinarian invoice notes that the medication should be consumed three times a day for three weeks from the date of purchase, the system 100 can provide notifications to the user device 106 three times per day to give the prescribed medicine to the pet.

The pet insurance module 120 also uses information in the user's profile, pet profile, and document module 118, among other modules, to generate a quote for the cost of the insurance for each pet. In one example embodiment, the system 100 presents a plurality of quotes for various scopes of protection for each pet of the user. The system can display the various quotes on a user interface for the user to select, accept, and/or decline the insurance coverage. If the user selects a quote for a protection scope, the system 100 can secure payment from the user to the insurance company directly. The system 100 can automatically upload the pet insurance policy documents from the pet insurance module 120 into the document module 118 upon selection and/or payment of the insurance policy.

The pet care management system 100 also enables users to submit insurance claims for processing through the pet insurance module 120. In one embodiment, the system 100 prompts the user to submit, upload, or otherwise provide a medical record and the invoice associated with the medical record. In some embodiments, the system 100 may requires that the medical records are provided directly by the health care provider. The system 100 analyzes the medical record, or the associated machine-readable code resulting from the OCR of the medical record and/or invoice and determines if additional information is needed.

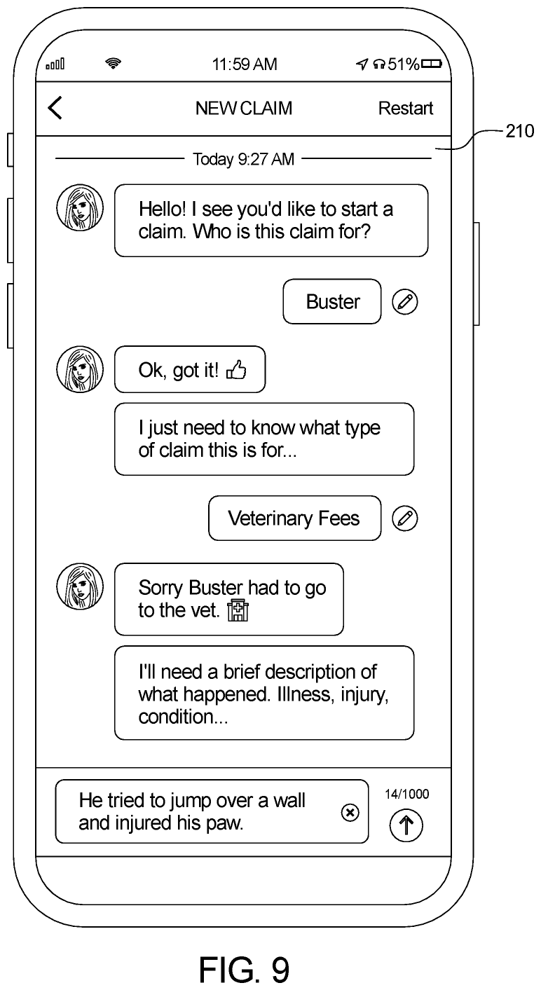

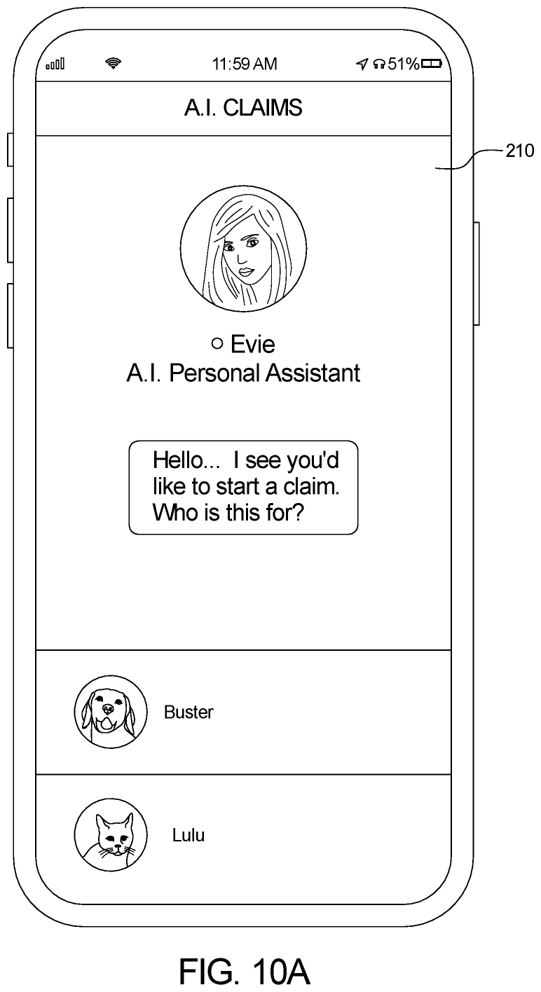

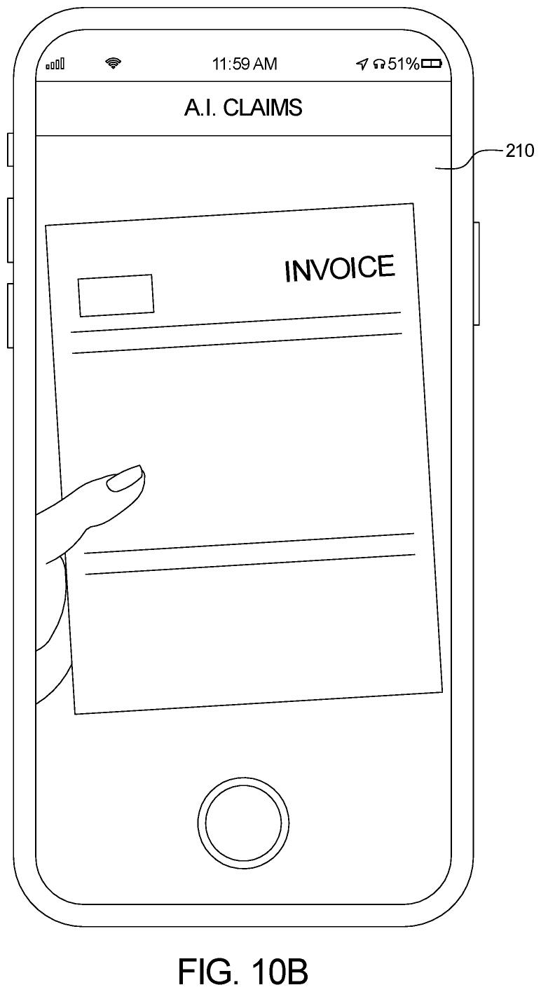

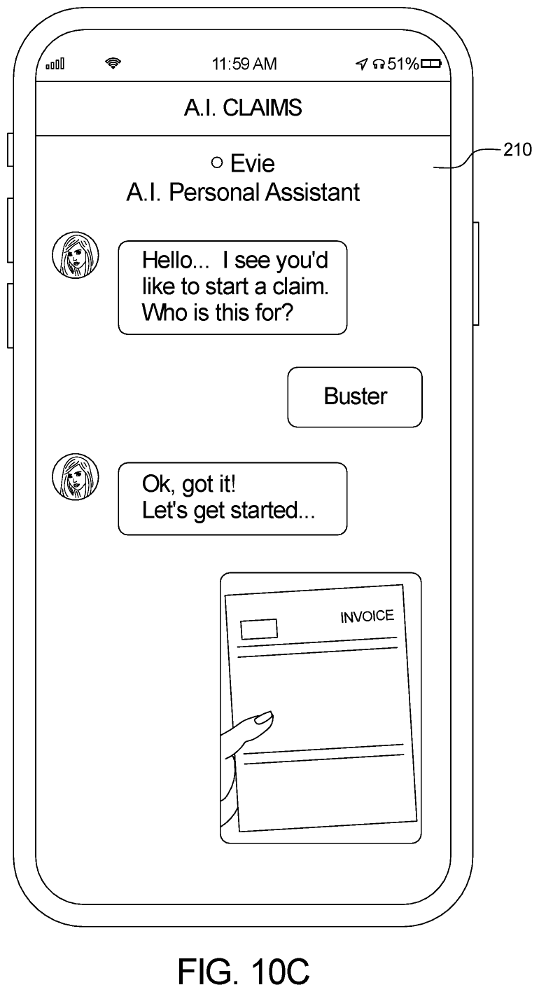

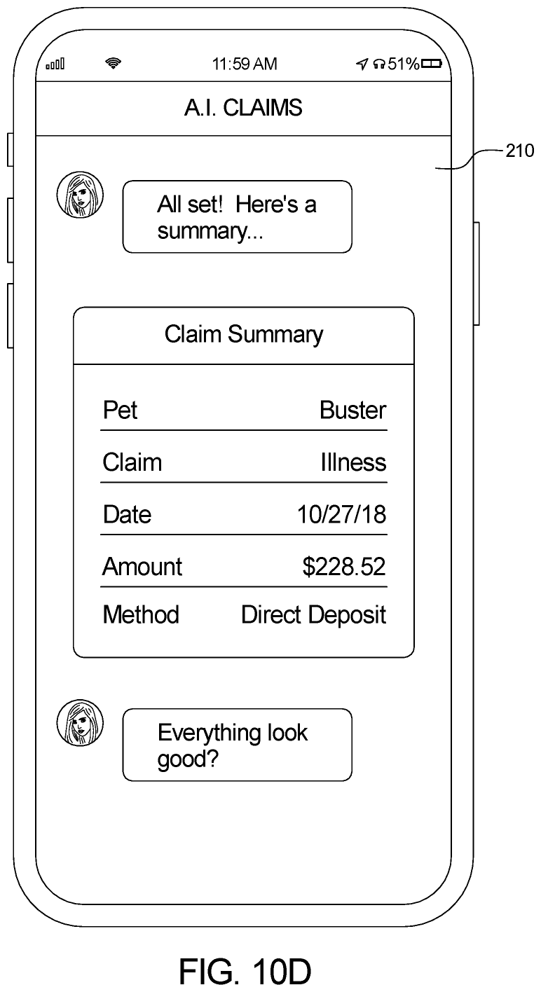

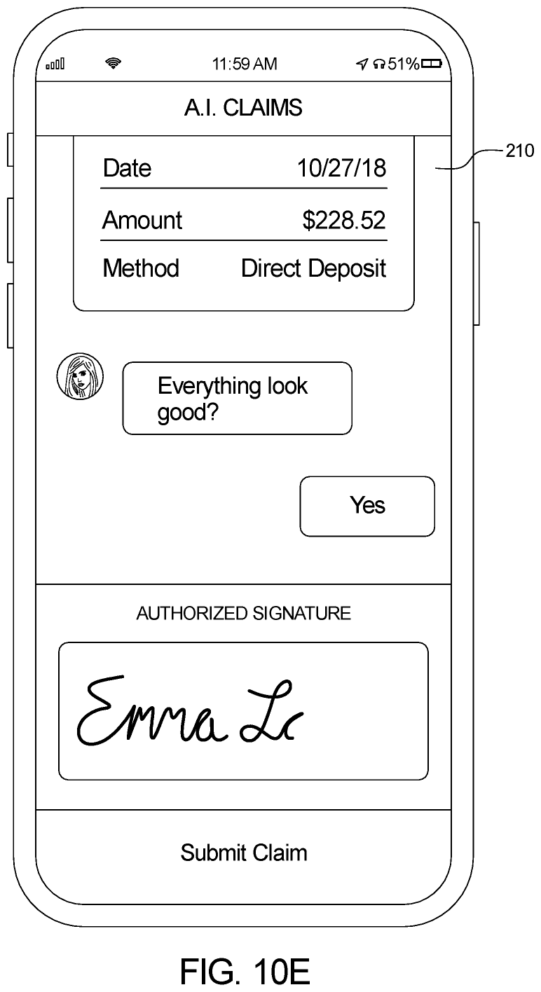

In one embodiment, the pet insurance module 120 collects additional information from the user through an interactive claims chat feature presented to the user through a claims user interface 210 shown in FIGS. 9 and 10A-10E. The chat feature may be a chat with a live representative or an automated system. Once the system 100 identifies what additional information is needed, the system 100 messages the user through the claims chat feature to collect specific details as shown in FIG. 8.

FIGS. 10A-10E illustrate user interfaces that show a chat conversation that a user may have with the system 100 via the chat feature of the pet insurance module 120 in order to process a claim. Upon opening the chat feature user interface 210, the system 100 prompts the user with questions to collect information needed to process the claim. In the illustrated embodiment, the system 100 first confirms the pet associated with the invoice at issue in the claim.

In one of the next steps, the system 100 then prompts the user to capture image data of the invoice or other document related to the insurance claim as shown in FIG. 10B. The image data is then submitted to the system 100 via the chat conversation as shown in FIG. 10C.

The system 100 converts the image data into machine-readable code and scans the document for information related to the claims processing. In one embodiment, the system 100 summarizes the relevant information and confirms the details with the user via the chat conversation as shown in FIG. 10D. Once confirmed, the system 100 presents a signature box to the user via the claims chat feature as shown in FIG. 10E. Once a signature has been entered, a submit claim button is provided to the user, enabling the user to submit the claim immediately.

As part of the claims processing, the system 100 can process the claim and reimburse the user's veterinarian invoice after deducting the co-pay from the total amount of the covered claim. The remaining balance is paid directly to the user's account electronically via the payment module 124. The system 100 may maintain a balance in a user account associated with the user. The user may use the funds in this account to pay other users and businesses for other services such as pet walking and pet day care. Alternatively, or in addition to, the system 100 can submit the reimbursement to the user's personal bank account or mail the payment to the user.

The social network module provides users with an interface to connect with other pet owners, share photos, plan play dates, and schedule events. The social network platform can connect a user's profile with a plurality of other user's profiles in a social feed. For example, the user can select and/or approve friend suggestions from the system to create the plurality of friend/family profiles. Each profile, depending on the user's security and preference conditions, can share a user's contact information, the user's pet(s) information, events the user is attending, businesses the user has used and/or reviewed, the pet park the user visits, the veterinarian the user uses, among others. In an example, the system can suggest friends for the user based on the user's profile, location, breed of pet, age of pet, among others. The system can allow a user to search for friends based on the breed of pets, location, veterinarian, among others.

The recommendation module allows users to explore pet friendly places and services such as pet parks, hiking trails, day care, restaurants, pet walkers, pet groomers, among others. The recommendation module can be included and/or in communication with the social network module. For example, the user can search for businesses in the user's location, restaurants that are pet friendly, pet activities in a certain distance from the user or a certain date/time range, among others, from the social network homepage. The system can suggest businesses, veterinarians, activities, events, etc. for the user based on the user's profile, pet profile, pet information, user location, among others. The user can also access the live veterinarian from the homepage for emergencies.

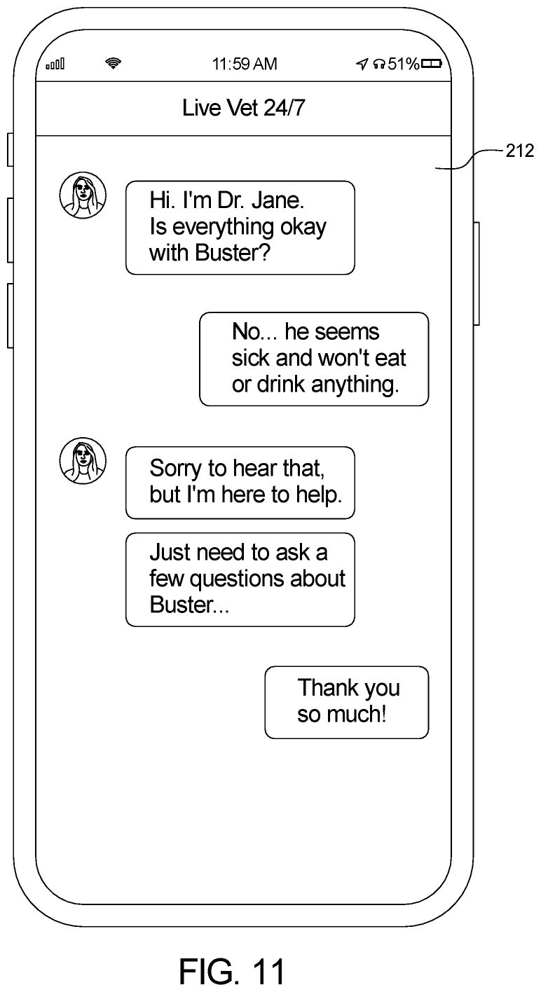

The veterinarian module 122 provides live access to a licensed veterinarian anytime via the vet user interface 212 shown in FIG. 11. In an example, the user communicates with a licensed veterinarian with any questions regarding a pet's health through email, instant messenger, live chat, or any other suitable messaging service. The present system enables user to contact a veterinarian immediately and directly to ask a question to and/or show the veterinarian images of the issues by a photo, video, live stream audio video, etc., for the veterinarian to answer and instruct the user on how to resolve the issue. This immediate interaction allows the user to avoid waiting to make an appointment with a veterinarian's office, waiting for the appointment, and traveling to the veterinarian's office.

The communication module 114 provides communications directly to other pet owners directly and through the user's social network, to veterinarians, and to businesses, among others, through an email server, video imaging, audio, among others. The communication system 114 also enables the user or the system 100 to deliver medical records to a veterinarian, a pet day care, dog walker, etc. In addition, the communication platform 114 can provide users with messages generated by the system to remind the user of various appointments, reminders, information, etc.

The payment module 124 can receive and store the user's payment information, such as the user name, credit card information, bank account details, etc. In an example, when the user selects and accepts an insurance policy, the system automatically completes payment of the policy to the insurance company from the user by way of the payment module 124. Similarly, the system 100 can secure payment from a user directly to a pet business, pet walker, pet groomer, pet day care, etc. For example, the system can complete payment of an appointment for the user's pet for a day care immediately upon the user confirming the appointment in the calendar module. Alternatively, instead of automatic payments, the user can select preferences such that the user submits payment from the payment module and/or another banking application to the recipient (e.g., pet business, pet daycare, pet insurance company, etc.).

The system can include a pet tracking module that communicates the location of the user's pet to the system controller. The system can track the pet's position as well as the amount of exercise, distance walked, sleeping time, among other things. The tracking data can be used in the pet insurance module 124, communicated to the veterinarian module 122, uploaded daily to the pet document module 118, etc. The tracking data can include or be independent of GPS technology. For example, the tracking module can be based on a gyroscope, accelerometer, among others to determine a pet's activity during a given time frame. The system can acquire the tracking data from a microchip within the pet, or a separate device that can attach to a pet's collar, for example.

In an example, the system can provide the user with a list of pets within a radius of the acquired pet location data. For example, the system can provide pet profiles of pets near the user, whether the pet profile is available for a pet play date, what times the pets near the user are available for play dates, etc.

The system can also collect user location data based on the user device. For example, the system can acquire pedometer data, GPS data, among others from the user device and associated time of movement. The system can analyze the user movement data and the pet movement data to determine whether the pet needs more exercise, whether the user or pet is sick from a decrease in movement, etc. For example, if a decrease in activity is found for the pet but not for the user, the system can predict a future insurance claim and intervene to provide care to the pet. Depending on the analysis, the system can suggest or automatically schedule a vet appointment, send notifications to the user that the pet needs an increase in movement (including a distance or time movement quota), a third party dog walker to increase the pet movement, etc. The system can also suggest products to the user based on the movement analysis of the pet and/or user. For example, if the system analyzes the movement data of the user and/or pet, the system may determine the activity decreases when the weather is colder than 20 degrees Fahrenheit. As such, the system can suggest products such as dog booties, dog coat, user hand-warmers, etc.

The pet's location can be used by the system to communicate the weather to a user. The daily weather can be sent as a notification to the user device based on the pet's location. In an example, the weather can be sent or displayed to the user device as a notification before the pet's usual walk or exercise time. For example, the system can automatically learn the times of day the pet usually goes for a walk based on historical GPS data. The system can analyze the GPS data to determine the time the pet usually goes on a walk, and the system can preemptively send a notification to the user the predicted weather for the usual walking time for that day.

The system can include a user credit function, wherein pet owners can have the ability to apply for credit to pay for veterinary expenses. The users can apply through the system and can have an instant response on how much credit they can receive. The funds associated with the user can be paid directly to the veterinarian to automatically pay for the services being provided. The system can include a soft credit function, and/or a repayment plan.

As mentioned above, aspects of the systems and methods described herein are controlled by one or more controllers. The one or more controllers may be adapted to run a variety of application programs, access and store data, including accessing and storing data in the associated databases, and enable one or more interactions as described herein. Typically, the controller is implemented by one or more programmable data processing devices. The hardware elements, operating systems, and programming languages of such devices are conventional in nature, and it is presumed that those skilled in the art are adequately familiar therewith.

For example, the one or more controllers may be a PC based implementation of a central control processing system utilizing a central processing unit (CPU), memory and an interconnect bus. The CPU may contain a single microprocessor, or it may contain a plurality of microprocessors for configuring the CPU as a multi-processor system. The memory may include a main memory, such as a dynamic random access memory (DRAM) and cache, as well as a read only memory, such as a PROM, EPROM, FLASH-EPROM, or the like. The system may also include any form of volatile or non-volatile memory. In operation, the memory stores at least portions of instructions for execution by the CPU and data for processing in accord with the executed instructions.

The one or more controllers may also include one or more input/output interfaces for communications with one or more processing systems. Although not shown, one or more such interfaces may enable communications via a network, e.g., to enable sending and receiving instructions electronically. The communication links may be wired or wireless.

The one or more controllers may further include appropriate input/output ports for interconnection with one or more output mechanisms (e.g., monitors, printers, touchscreens, motion-sensing input devices, etc.) and one or more input mechanisms (e.g., keyboards, mice, voice, touchscreens, bioelectric devices, magnetic readers, RFID readers, barcode readers, motion-sensing input devices, etc.) serving as one or more user interfaces for the controller. For example, the one or more controllers may include a graphics subsystem to drive the output mechanism. The links of the peripherals to the system may be wired connections or use wireless communications.

Although summarized above as a PC-type implementation, those skilled in the art will recognize that the one or more controllers also encompasses systems such as host computers, servers, workstations, network terminals, and the like. Further one or more controllers may be embodied in a device, such as a mobile electronic device, like a smartphone or tablet computer. In fact, the use of the term controller is intended to represent a broad category of components that are well known in the art.

Hence aspects of the systems and methods provided herein encompass hardware and software for controlling the relevant functions. Software may take the form of code or executable instructions for causing a controller or other programmable equipment to perform the relevant steps, where the code or instructions are carried by or otherwise embodied in a medium readable by the controller or other machine. Instructions or code for implementing such operations may be in the form of computer instruction in any form (e.g., source code, object code, interpreted code, etc.) stored in or carried by any tangible readable medium.

As used herein, terms such as computer or machine “readable medium” refer to any medium that participates in providing instructions to a processor for execution. Such a medium may take many forms. Non-volatile storage media include, for example, optical or magnetic disks, such as any of the storage devices in any computer(s) shown in the drawings. Volatile storage media include dynamic memory, such as the memory of such a computer platform. Common forms of computer-readable media therefore include for example: a floppy disk, a flexible disk, hard disk, magnetic tape, any other magnetic medium, a CD-ROM, DVD, any other optical medium, punch cards paper tape, any other physical medium with patterns of holes, a RAM, a PROM and EPROM, a FLASH-EPROM, any other memory chip or cartridge, or any other medium from which a controller can read programming code and/or data. Many of these forms of computer readable media may be involved in carrying one or more sequences of one or more instructions to a processor for execution.

It should be noted that various changes and modifications to the embodiments described herein will be apparent to those skilled in the art. Such changes and modifications may be made without departing from the spirit and scope of the present invention and without diminishing its attendant advantages. For example, various embodiments of the systems and methods may be provided based on various combinations of the features and functions from the subject matter provided herein.

We claim:

1. A pet care management system for scheduling a pet care appointment based on a medical record for a pet, the pet management system comprising:
a user device including a camera subsystem;
a database storing a calendar associated with the pet;
a processor in communication with the user device and the database;
memory in communication with the processor, memory storing computer executable instructions such that, when the instructions are executed by the processor, the processor is configured to:
capture, via the camera subsystem on the user device, image data representing the medical record related to a pet health care provider appointment, wherein the image data includes a date associated with veterinary services provided to the pet;
determine an appointment date for a subsequent appointment based on the date associated with veterinary services; and
automatically populate the calendar with the subsequent appointment on the appointment date.

2. The pet care management system of claim 1, wherein the processor is further configured to convert, via optical character recognition technology, the image data into a machine-readable code, wherein the machine-readable code includes the date associated with veterinary services.

3. The pet care management system of claim 1, wherein the medical record is one of a veterinarian visit record, a vaccination certificate, a vaccination history, a medication history, a surgery record, a hospital visit record, an insurance claim, and a veterinarian invoice.

4. The pet care management system of claim 1, wherein the database includes a plurality of time frames and a plurality of veterinary services types, wherein one of the plurality of time frames is associated with each one of the plurality of veterinary services types, and
wherein the step of determining an appointment date for the subsequent appointment includes the steps of:
identifying a veterinary services type associated with the veterinary services; and
selecting the appointment date of the subsequent appointment by adding the time frame associated with the veterinary services type of the veterinary services to the date associated with the veterinary services.

5. The pet care management system of claim 4, wherein each time frame comprises a preset number of weeks, months, or years.

6. The pet care management system of claim 4, wherein the veterinary services types includes at least one of a rabies vaccination, a hepatitis vaccination, a canine parvovirus vaccination, a canine distemper vaccination, a Bordetella vaccination, a canine influenza vaccination, a leptospirosis vaccination, a lyme vaccination, a feline panleukopenia vaccination, feline viral rhinotracheitis vaccination, feline caliciviral disease vaccination, a feline chlamydiosis vaccination, a feline leukemia virus vaccination, a feline infection peritonitis vaccination, a bordetellosis vaccination, a ringworm vaccination, and a giardiasis vaccination.

7. The pet care management system of claim 4, wherein the veterinary services types includes an ear infection, a behavioral consultation, dental care, parasite control, and neutering.

8. The pet care management system of claim 1, wherein the veterinary services comprises one of a procedure, a date of surgery, a date of treatment, a date of an incident related to the health of the pet, and a date of release from a pet hospital.

9. The pet care management system of claim 1, wherein the machine-readable code includes a follow-up appointment date associated with the date associated with the veterinary services, and wherein the step of determining an appointment date for the subsequent appointment comprises selecting the follow-up appointment date as the appointment date.

10. The pet care management system of claim 1, wherein the machine-readable code includes a follow-up appointment time frame associated with the date associated with the veterinary services, and wherein the step of determining an appointment date for the subsequent appointment comprises adding the follow-up appointment time frame to the date associated with the veterinary services.

11. The pet care management system of claim 1, wherein the processor is further configured to automatically generate a reminder to schedule the subsequent appointment with a pet health care provider associated with the veterinary services.

12. The pet care management system of claim 1, wherein the processor is further configured to automatically schedule the subsequent appointment with a pet health care provider associated with the veterinary services.

13. The pet care management system of claim 1, wherein the processor is further configured to automatically generate notifications related to payment to a pet health care provider associated with the veterinary services.

14. A method for scheduling a pet care appointment based on a medical record for a pet through a user device, the method comprising the steps of:
providing a calendar associated with the pet through a user interface on the user device;
receiving image data from the user device, wherein the image data is captured through camera functionality on the user device, and wherein the image data represents the medical record related to a pet care provider appointment;
determining an appointment date for a subsequent appointment based on the date associated with veterinary services; and
automatically populating the calendar with the subsequent appointment on the appointment date.

15. The method of claim 14, further comprising the step of:
converting, via optical character recognition technology, the image data into a machine-readable code, wherein the machine-readable code includes the date associated with veterinary services.

16. The method of claim 14, further comprising the step of accessing a database including a plurality of time frames and a plurality of veterinary services types, wherein one of the plurality of time frames is associated with each one of the plurality of veterinary services types; and
wherein the step of determining an appointment date for the subsequent appointment includes the steps of:
identifying a veterinary services type associated with the veterinary services; and
selecting the appointment date of the subsequent appointment by adding the time frame associated with the veterinary services type of the veterinary services to the date associated with the veterinary services.

17. The method of claim 14, further comprising the step of:
automatically scheduling the subsequent appointment with a pet health care provider associated with the veterinary services.

18. The method of claim 14, further comprising the step of:
automatically generating notifications related to payment to a pet health care provider associated with the veterinary services.
